(19) United States
(12) Patent Application Publication
(10) Pub. No.: US 2011/0118588 A1
(43) Pub. Date: May 19, 2011

(54) Combination MRI and Radiotherapy Systems and Methods of Use

(76) Inventors: Giora Komblau, Binyamina (IL); David Maier Neustadter, Nof Ayalon (IL); Saul Stokar, RaAnana (IL)

(21) Appl. No.: 12/922,398

(22) PCT Filed: Mar. 12, 2009
(86) PCT No.: PCT/IL2009/000278

§ 371 Date: Dec. 27, 2010

Related U.S. Application Data
(60) Provisional application No. 61/069,277, filed on Mar. 12, 2008.

(51) Int. Cl. A61B 5/055; A61N 5/00

(52) U.S. Cl. 600/411

(57) ABSTRACT

A combination MRI and radiotherapy system comprising: a) an MRI system for imaging a patient receiving radiotherapy, comprising a magnetic field source suitable for generating a magnetic field of strength and uniformity useable for imaging, capable of being ramped up to said magnetic field in less than 10 minutes, and ramped down from said magnetic field in less than 10 minutes; b) a radiation source configured for applying radiotherapy; and c) a controller which ramps the magnetic field source down to less than 20% of said magnetic field strength when the radiation source is to be used for radiotherapy, and ramps the magnetic field source up to said magnetic field strength when the MRI system is to be used for imaging.

### RELATED APPLICATION/S

The present application claims benefit under 35 USC 119(e) from U.S. provisional patent application 61/069,277, filed on Mar. 12, 2008.

The contents of the above document are incorporated by reference as if fully set forth herein.

### FIELD AND BACKGROUND OF THE INVENTION

The present invention, in some embodiments thereof, relates to combination radiotherapy and MRI systems and methods of using them, and more particularly, but not exclusively, to combination systems in which the main MRI magnet can be quickly ramped up for imaging and down for radiotherapy.

In radiation therapy (also known as radiotherapy), ionizing radiation is used to destroy tissues affected by proliferative tissue disorders such as cancer. In external beam radiotherapy, a radiation source is placed outside the body of the patient and the target within the patient is irradiated with an external radiation beam. Two types of external radiation sources are typically used—radiation produced by radioactive sources (radionuclides such as ⁶⁰Co) and radiation produced artificially using a medical linear accelerator (or “linac”). Although ⁶Co teletherapy was in the forefront of radiotherapy for a number of years, in recent years medical linacs have largely superseded ⁶⁰Co machines.

As used herein, the term “linac” refers to linear accelerators used for radiation therapy treatment, as well as to any other source that emits pulses (or intermittent controlled amounts) of ionizing radiation or of ionizing particles. The physical principles of linear accelerators are well known. See, for instance, Principles of Charged Particle Acceleration by Stanley Humphries (ISBN 0-471-87878-2) or the Fermilab Operations Rookie Books, available on the World Wide Web as an online book at: www-bdnewinal.gov/operationshookie_books/rbooks.html, in particular Accelerator Concepts v3.1 (Nov. 15, 2006), and Linac Rookie Book v2.1 (Oct. 1, 2004), both downloaded on Mar. 12, 2009.

In an electron linac, electrons are accelerated to high energies (typically 4-25 MeV) and aimed at an X-ray target. As the beam of electrons is decelerated in the target, a beam of high energy bremsstrahlung photons is emitted. These high energy photons are also known as X-rays or gamma rays, although the latter term is usually reserved for the photons emitted by a nuclear transition, such as those emitted by a radioactive source like ⁶⁰Co.

Radiotherapy involving x-ray radiation often utilizes a device containing an X-ray or gamma ray source, such as a linac or a radionuclide source, having a head that is mounted on a mechanical structure known as a gantry. The head may be rotated about the patient's long axis. A set of wedges, collimators, and filters manipulate the X-ray linac beam so that it has the spatial and energy profile optimal for treating the target tissue, e.g., a tumor. Typically, the linac head is rotated around a patient's superior-inferior axis during a treatment session through a set of gantry angles (or “fields”) selected to maximize the dose accumulated by the target tumor tissue and to minimize the dose deposited in healthy tissue. In the target volume the beams from different directions intersect and accumulate a large cumulative dose. Other tissues outside of the target volume accumulate a smaller dose.

Radiation planning has, as an important goal, the avoidance (as much as possible) of harming healthy tissue. A high resolution 3D image, e.g., a computerized tomography (CT) data set, of the involved anatomy is used in properly planning the gantry angles and doses to be provided at each field. Magnetic resonance imaging (MRI), ultrasound, and nuclear imaging are also used. Such image sets are often obtained from independent CT or MRI systems. Such imaging systems are not mechanically connected to the radiation therapy system.

Once the radiation distribution is planned, the radiotherapy treatment can begin. However, for the radiation planning to be most accurate and relevant, the patient should be positioned relative to the linac beam in exactly the same orientation and position as in the radiation planning images. Even if the patient is positioned relative to the linac in the same supine position (head in, face up) as during the planning CT step, the patient may still not be in the exact same positioning on the bed of the radiotherapy system. For instance, the patient may be slightly rotated or translated, the bed may have a different shape, or the bed may not be oriented or positioned in exactly the same way relative to the axes of the linac, as it was relative to the axes of the imaging system used for radiation planning.

Accuracy of patient positioning is important, and it is quite difficult to attain adequate accuracy, since many internal organs seen during the CT planning scan (e.g. the prostate) are not visible or palpable from outside the body. Further, certain of the internal organs themselves move relative to the body's bony markers; for example, the prostate moves as the bladder fills and empties, the lungs and the liver (along with any tumors in those organs) move as the lungs fill and empty and as the heart beats.

This requirement for accuracy in patient positioning poses a difficulty, in that commercial linac systems generally do not include built-in CT or MRI scanners.

Methods that may be used to verify treatment position include the use of external markers or “fiducials” such as natural references (e.g., bones) or artificial markers such as skin-borne tattoos. Methods using external markers are simple but their accuracy is low, using, as they do, the assumption that the external marker remains at the same position and orientation relative to the tumor throughout the entire radiotherapy regimen. The assumption is poor for soft-tissue tumors whose positions can change from day to day, from hour to hour, and even from minute to minute. The assumption is poor for certain internal organs such as the prostate gland (and any associated tumor) since the spatial relationship between the organ and the external marker will vary depending on the volume of the bladder. The use of external fiducial markers is even less accurate in areas of the body where breathing motion affects the tissue in question, e.g., in lung or liver tumors.

Megavoltage imaging (using the treatment beam to produce a crude CT-like image) produces low quality images in which it is often not possible to distinguish soft tissues. To overcome this problem, radio opaque markers, such as gold seeds, may be injected into a tumor at the start of the treatment regime. In this method, the offset of the gold seeds from the tumor isocenter is measured during radiation planning. At the start of each radiation session a megavolt image is obtained and the gold spheres positions are used to position the patient for irradiation. Another source of positioning inaccuracy is due to the changes in tumor geometry over the course of the treatment. As the tumor shrinks during the course of radiotherapy, it may be medically advantageous to adjust the doses to conform to the shape of the shrinking tumor. However, the costs of repeatedly imaging the tumor are often quite high, as are the costs, in time and manpower, of recalculating the radiation dose. In any case, imaging techniques that do not show the soft tissue cannot provide such information.

MRI is a widely regarded choice for diagnostic imaging in many parts of the body. A review of the theory and practice of MRI is given in E. M. Haacke, R. W. Brown, M. R. Thompson, R. Venkatesan, Magnetic Resonance Imaging—Physical Principles and Sequence Design, Wiley-Liss, NY (1999), as well as in U.S. Pat. No. 5,835,995 to Macovski and Conolly. MRI is particularly useful in providing high resolution imaging of soft tissue, particularly those of the central nervous system, and in distinguishing different types of soft tissue, including distinguishing tumors from healthy tissue. In addition, MRI allows (in principle) for measurement of tissue parameters other than mere gross anatomy, such as blood flow, diffusion, temperature, and functionality. MRI is an excellent choice for initial treatment planning and would be a similarly excellent choice for positioning the patient at the linac. However, when MRI images are obtained at a separate location, with the images transferred to a radiation-planning computer, the potential for inaccuracy remains.

Prepolarized MRI is a variation of MRI. It is motivated by the idea that a uniform static magnetic field plays two roles in MRI—it creates a longitudinal magnetization that is the source of the MR signal, and it is the source of the Lanuor procession that generates the free induction decay (FID) signal. In 1993, Makovski and Conolly proposed a new MRI technique, known as prepolarized MRI (PMRI) or sometimes as field-cycled MRI See, for example, A. Macovski, S. Conolly, Novel Approached to Low-Cost MRI, Mag. Res. Med. 30, 221-230 (1993); P. Morgan, S. Conolly, G. Scott and A. Makovski, A Readout Magnet for Prepolarized MRI, Mag. Res. Med. 36, 527-536 (1996); U.S. Pat. No. 5,057,776 to Macovski; Tina Pavlin, Hyperpolarized Gas Polarimetry and Imaging at Low Magnetic Field (Cal Tech PhD thesis, 2003), available at: etd.caltech.edu/etd/available/etd-05302003-134718/unrestricted/00_master_file.pdf (Chapter 3, “The Pulsed Resistive Low-Field MR Scanner,” contains a review of PMRI theory and hardware), downloaded on Oct. 24, 2007; and Commission on Physical Sciences, Mathematics, and Applications, Mathematics and Physics of Emerging Biomedical Imaging, National Academy of Sciences ISBN 978-0-309-05387-7 (1996), section 4.2.2 (Pulsed-field MRI systems), available at: www.nap.edu/openbook.php?isbn=0309053870.

Macovski and Connoly noted that the two roles of the magnetic field make different demands on the system. The first role of the magnetic field has only mild homogeneity requirements. A uniformity of 10%, for example, may be adequate. The second role of the uniform magnetic field generally requires an extremely uniform field, for example at the part per million level or better, but does not require very high fields, and there may even be advantages to using fields that are not too high. Standard MRI systems, in which a high field is present both during the signal excitation and during the signal readout, suffer from increased artifacts from inhomogeneity, susceptibility, and chemical shifts, as compared with low field systems.

Makovski and Conolly's PMRI system includes two independent, co-axial magnets, the first (the “polarizing magnet”) being a pulsed, high-field magnet of limited homogeneity and the second being a highly homogeneous, low-field magnet used for signal readout. The readout magnet may be a superconducting or permanent magnet. The polarizing magnet is resistive to allow pulsing the magnet on and off, since the polarizing magnet must be pulsed off during signal readout to avoid contaminating the MRI signal due to its poor homogeneity. Typically, the polarizing magnet pulses on in up to a second and pulses off as rapidly as possible, often in less than 100 msec. In most implementations of PMRI, the polarizing magnet is resistive, due to the technical difficulty of pulsing a superconducting magnet as well as the increased cost of superconducting magnets relative to resistive magnets.

Combined medical linac and MRI systems are described by B. Raaymakers, A. Raaijmakers, A. Kotte, D. Jette, and J. Lagendijk, Integrating a MRI scanner with a 6 MV radiotherapy accelerator: dose deposition in a transverse magnetic field, Phy. Med. Bio. 49 (2004) 4109-4118; A. Raaijmakers, B. Raaymakers, and J. Lagendijk, Integrating a MRI scanner with a 6 MV radiotherapy accelerator: dose increase at tissue-air interfaces in a lateral magnetic field due to returning electrons, Phy. Med. Bio. 50 (2005) 1363-1376; A. Raaijmakers, B. Raaymakers, S. van der Meer, and J. Lagendijk, Integrating an MRI scanner with a 6 MV radiotherapy accelerator: impact of the surface orientation on the entrance and exit doses due to the transverse magnetic field, Phy. Med. Bio. 52 (2007) 929-939; A. Raaijmakers, B. Raaymakers, and J. Lagendijk, Experimental verification of magnetic field dose effects for the MRI-accelerator, Phy. Med. Bio. 52 (2007) 4283-4291. These papers point out problems introduced by the linac and the MRI system interfering with each other. The problems include:

1) The components of the MRI system form a physical barrier to the linac's radiation beam, attenuating and scattering the beam.
2) The magnetic field created by the MRI system usually extends beyond the physical volume of the MRI system, and any such external magnetic fields imposed upon the linac may adversely affect the electron beam used to create the linac's radiation, by changing the path of the electron beam so it is not accelerated properly, or misses its target.
3) A magnetic field imposed upon the patient skews the radiation dose distribution within the patient, due to its effect on secondary electrons produced inside the patient by the incident x-rays or gamma rays, especially in low density organs such as the lungs. The problem of calculating the dose distribution is made more difficult by the fact that the magnetic field is inhomogeneous inside the body, due to the magnetic susceptibility of the body. It is very difficult to model or measure this inhomogeneity accurately in-vivo and therefore it is very difficult to take it into account during radiation planning.
4) The RF section of the linac, used for accelerating the electron beam, introduces substantial noise into the MRI image, especially if the Larmor frequency of the MRI magnetic field is near an RF frequency used by the linac, or a harmonic of it.
5) Ferromagnetic components of the linac distort the magnetic field in the neighborhood, leading to artifacts and loss of resolution on the MRI image. Compensating for the field distortion is difficult because the linac typically is on a gantry that moves relative to the MRI system.

U.S. Pat. Nos. 6,198,957 and 6,366,798 (to Green, each assigned to Varian), describe an MRI system that allows for simultaneous acquisition of an MR image and radiotherapy treatment. The MR magnet has an open ring configuration (i.e. it has the form of a double doughnut—see FIG. 2) to allow unimpeded access for the radiotherapy beam. Published PCT Application No. WO 03/008986 (Lagendijk and Wouter, assigned to Elekta) also describes a system wherein the MRI has an open ring configuration. These devices only overcome the first problem listed above.

Published PCT Application WO 2004/024235 (Lagendijk and Wouter, assigned to Elekta) also describes a combined linac and MRI system. In order to avoid the adverse effect of the MRI field on the linac, this publication teaches the use of an actively shielded magnet, having a highly reduced fringe (or exterior) field. Active shielding may be accomplished by surrounding the first magnet with another magnet (collinear with the first cylinder) whose function is to cancel the net field outside the magnet pair. Such actively shielded magnets are well-known in the MRI field, since they accomplish the goal of shielding the outside world from the effects of the strong magnetic field. In the case of an MRI system integrated with a linac, this effect is accomplished by providing shielding so that the magnetic field in the doughnut-shaped volume through which the linac head traverses is substantially zero. This method does not address the third problem mentioned above, i.e., the effect of the magnetic field on the target tissue dose.

Published PCT Application WO 2006/097274 (to Raaymakers and Lagendijk, assigned to Elekta) presents methods that ensure that the RF coils do not substantially interfere with the radiotherapy beam. This procedure addresses only the second problem listed above.

Published PCT Application WO 2007/045076 (to Fallone, Carlone, and Murray, assigned to Alberta Cancer Board) discusses a combined MRI and radiotherapy system in which the relative orientation of the MRI magnet and the gantry is fixed (i.e. both rotate together around the patient). As a result, there is no change in magnet field homogeneity, during gantry rotation around the patient. To solve the problem of the linac's RF interfering with the MRI system, the publication discusses utilizing the fact that the linac only produces RF (and hence RF interference) in bursts, and the MRI system is only sensitive to RF noise within specific time windows. Synchronizing or interleaving these two avoids this interference (see FIG. 3 for a timing diagram) and can therefore help overcome this problem. In this arrangement, the MRI acquisition window and the linac irradiation pulse have only short (1-100 millisecond) time shifts between them.

Published U.S. Application 2005/0197564 (to Dempsey, assigned to Univ. of Florida Research Foundation) discloses using radioisotopes (including ⁶⁰Co) as radiation sources in a combined MRI-radiotherapy system. Since the ⁶⁰Co source does not accelerate electrons to produce radiation, there is no need to shield it from the magnetic field of the MRI system. Dempsey discloses using a low field MRI system, since the effect of the magnetic field on the spatial distribution of the radiation dose is decreased at low field. However, low field MRI systems have the disadvantage that they require longer acquisition time than high field MRI systems, for the same signal-to-noise ratio and pixel size. This could result in inefficient use of the expensive radiotherapy system if much more time is spent acquiring images than is spent irradiating the patient, and the longer treatment sessions may be more uncomfortable for the patient.

U.S. Pat. No. 6,862,469 (to Bucholtz and Miller, assigned to St. Louis University) discloses a proton therapy system in combination with an MRI system. The MRI system monitors the 3D position of the tumor and activates the proton beam only when the tumor is within the planned volume.

Resistive magnets were widely used in MRI in the 1970s and early 1980s. See, for example, Resistive and Permanent Magnets for Whole Body MRI, Frank Davies, in Encyclopedia of Magnetic Resonance, John Wiley and Sons, 2007, DOI: 0.1002/9780470034590.emrstm0469. Resistive magnets fell from favor in MM during the 1980's when the trend in MRI turned to high field systems. This trend had several impetuses. Whole-body resistive MM magnets having a field strength above about 0.35 T are difficult to fabricate. The heat generated in the magnet coils is not easily dispersed. The currents in resistive magnet coils are not readily stabilized at the level required for MRI. This latter difficulty increases with increasing magnet current (i.e., increasing magnetic field strength). See, U.S. Pat. No. 5,570,022, to G. Enfold, S. Pekoe and J. Virtanen, entitled Power Supply for MRI Magnets.

### SUMMARY OF THE INVENTION

An exemplary embodiment of the invention concerns a combined MRI and radiotherapy system, in which an MRI magnetic field is ramped up for imaging, but is ramped down for radiotherapy.

There is thus provided, according to an exemplary embodiment of the invention, a combination MRI and radiotherapy system comprising:
a) an MRI system for imaging a patient receiving radiotherapy, comprising a magnetic field source suitable for generating a magnetic field of strength and uniformity useable for imaging, capable of being ramped up to said magnetic field in less than 10 minutes, and ramped down from said magnetic field in less than 10 minutes;
b) a radiation source configured for applying radiotherapy; and
c) a controller which ramps the magnetic field source down to less than 20% of said magnetic field strength when the radiation source is to be used for radiotherapy, and ramps the magnetic field source up to said magnetic field strength when the MRI system is to be used for imaging.

Optionally, the magnetic field source comprises a non-superconducting coil.

In an exemplary embodiment of the invention, the MRI system comprises a prepolarized MRI system, and the magnetic field source comprises a high field source for polarization and a low field source for readout.

Optionally, the high field source comprises a superconducting coil capable of ramping up to a high magnetic field used for polarization, and ramping down from the high magnetic field, each in less than 10 minutes.

Optionally, the low field source comprises a superconducting coil.

Optionally, the superconducting coil of the low field source is capable of ramping up to a low magnetic field used for readout, and ramping down from the low magnetic field, each in less than 10 minutes.

Optionally, the controller controls the MRI system not to acquire images when the radiation source is being used for radiotherapy.

In an exemplary embodiment of the invention, the system comprises a real-time tracker which tracks changes in position of a radiotherapy target in the patient.

Optionally, the tracker includes a radioactive marker.

Alternatively or additionally, the tracker comprises an image-based tracking system.

Alternatively or additionally, the tracker comprises an implanted leadless marker.

Optionally, the radiation source comprises a linac.

Alternatively or additionally, the radiation source comprises a radioactive source.

Optionally, the magnetic field source of the MRI system comprises an open magnet.

Optionally, the magnetic field source is sufficiently well shielded magnetically such that the magnetic field used for imaging is less than 100 gauss throughout any volume where the radiation source is located during imaging.

Optionally, the controller and magnetic field source are configured such that when the controller ramps the magnetic field source down, the magnetic field is less than 100 gauss in any volume in which the system is configured to receive part of the body of the patient during radiotherapy.

Optionally, the magnetic field used for imaging reaches at least 1 tesla, throughout an imaging region, when the magnetic field source is ramped up.

There is further provided, according to an exemplary embodiment of the invention, a method of radiotherapy of a target volume in a patient, comprising:
a) ramping up a magnet of an MRI system in less than 10 minutes;
b) acquiring one or more MRI images of the target volume, using the ramped up magnetic field;
c) ramping down the magnet to a field lower by at least a factor of 5, in less than 10 minutes, after using the field for the MRI imaging; and
d) applying radiotherapy radiation from a radiation source to the target volume with the magnet ramped down, taking into account the position of the target volume as indicated in the MRI images, while keeping the radiation source registered to the MRI system between acquiring the images and applying the radiation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volitile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 schematically shows a diagram of the pulse sequence for prepolarized MRI, according to the prior art;

FIG. 2 shows a schematic of a device having an open coil MRI, according to the prior art;

FIG. 3 schematically shows a prior art timing diagram for a combined MRI and linac system;

FIG. 4 schematically shows a combined MRI and radiotherapy system, according to an exemplary embodiment of the invention;

FIG. 5 shows a flow diagram for a method of using a combined MRI and radiotherapy system according to an exemplary embodiment of the invention; and

FIG. 6 shows a flow diagram for a method of using a combined MRI and radiotherapy system, according to another exemplary embodiment of the invention.

### DESCRIPTION OF EMBODIMENTS OF THE INVENTION

The present invention, in some embodiments thereof, relates to combination radiotherapy and MRI systems and methods of using them, and more particularly, but not exclusively, to combination systems in which the main MRI magnet can be quickly ramped up for imaging and down for radiotherapy. Alternating periods when the field has been ramped up, and MRI images are acquired, with periods when the field has been ramped down, and doses of radiation are applied to a tumor or other target in a patient, may allow nearly real-time MRI images of the target, and hence more accurate application of radiation, while avoiding the problems, listed above, that make it difficult to acquire MRI images during the application of radiation to a patient.

An exemplary embodiment of the invention concerns a combined MRI and radiotherapy system, in which an MRI magnetic field source is ramped up to a magnetic field used for imaging, and ramped down to a much weaker field, or to zero magnetic field, for radiotherapy of a patient. The magnetic field used for imaging, in all or in part of the region being imaged, is optionally greater than 3 tesla, between 2 and 3 tesla, between 1 and 2 tesla, between 0.5 and 1 tesla, between 0.35 and 0.5 tesla, between 0.1 and 0.35 tesla, or less than 0.1 tesla. The weaker magnetic field is optionally at least 10 times weaker than the field used for imaging, and is optionally less than 1000 gauss, or less than 100 gauss. The magnetic field is ramped up and ramped down in times less than 10 minutes each, optionally less than 5 minutes, less than 2 minutes, less than 1 minute, or less than 30 seconds, allowing the position of a tumor or other radiotherapy target to be determined from the MRI images in nearly real time. Optionally, the ramping time is shorter than the time of radiotherapy for each irradiation field, or shorter than 5 times, 2 times, 0.5 times, or 0.2 times the time of radiotherapy for each irradiation field. Optionally, the ramping time is shorter than the total time of radiotherapy during a treatment session, or shorter than 50%, 20%, 10%, or 5% of the total time of radiotherapy. The absence of a strong magnetic field, or of any magnetic field, during the radiotherapy, avoids the problem of calculating the radiation dose in the presence of a magnetic field, as well as adverse effects of the magnetic field on a radiation source for the radiotherapy, if it is an accelerator for example.

Optionally, no images are acquired when the radiotherapy is being performed, avoiding RF interference to the imaging if the radiation source is an accelerator which uses RF fields. Optionally, the MRI magnetic field source is sufficiently well shielded magnetically so that any stray fields, reaching the radiation source, are weaker than 100 gauss, or weaker than 30 gauss, 10 gauss, 3 gauss, or 1 gauss. Optionally, these limits on the stray magnetic field apply at least to any part of the radiation source where an electron beam is present, in the case of a linac radiation source, even if they do not apply to the entire structure of the radiation source. Additionally or alternatively, these limits on the stray magnetic field apply at least to any part of the radiation source that is ferromagnetic. With such weak fields, any magnetization of ferromagnetic materials in the radiation source has negligible effect on the delivery of radiation, in the case of an accelerator for example, and any distortion of the MRI magnetic field, due to ferromagnetic materials in the radiation source, has negligible effect on the image quality.

Optionally, the MRI magnetic field source comprises an open MRI magnet, allowing the radiation source access to the patient, without significant scattering or attenuation of radiation by the magnet or other parts of the system, and without radiation from the radiation source significantly damaging the MRI system.

In some embodiments of the invention, the MRI magnetic field source is a non-superconducting magnet, which can be quickly ramped up and down. Alternatively, the MRI magnetic field source is a superconducting magnet of a design which can be ramped up and down at least relatively quickly, for example with any of the ramping times listed above. In some embodiments of the invention, an MRI portion of the combined MRI and radiotherapy system comprises a prepolarized MRI (PMRI) system, and the MRI magnetic field source comprises a high field source generating a high magnetic field, not necessarily very uniform, for polarizing the nuclei in a region being imaged, and a low field source, generating a low and very uniform magnetic field, for readout of the image, after the high field source has been ramped down. Optionally, both the high and low field sources can be ramped up and down relatively quickly. Alternatively, only the high field source can be ramped up and down quickly, and the low field source remains on during radiotherapy, for example if it produces a magnetic field that is weak enough to have a negligible effect on the radiation dose distribution, and optionally weak enough to have a negligible effect on the path of any electron or ion beam produced by the radiation source. The polarizing magnetic field is optionally greater than 3 tesla, between 2 and 3 tesla, between 1 and 2 tesla, between 0.5 and 1 tesla, between 0.35 and 0.5 tesla, between 0.1 and 0.35 tesla, or less than 0.1 tesla. The readout magnetic field is optionally greater than 1000 gauss, or between 500 and 1000 gauss, or between 100 and 500 gauss, or less than 100 gauss. The readout field is less than the polarizing field, optionally less than 50% of the polarizing field, or less than 20%, or less than 10%.

In some embodiments of the invention, the system comprises a tracker which can track the position of a radiotherapy target, for example a tumor in the patient, providing information about the position of the target in real time, including intervals between acquisition of MRI images, for example during the application of radiotherapy when the MRI magnetic field source is ramped down. The tracker is, for example, an RF tracker, a tracker using a radioactive marker or an implanted leadless marker, or an image-based tracker, using for example an imaging modality other than MRI. Leadless markers are sometimes called wireless markers, and as used herein, the terms “leadless marker” and “wireless marker” are synonymous.

In some embodiments of the invention, a radiotherapy radiation source of the combined system comprises a linear accelerator (linac). In some embodiments of the invention, the radiation source comprises a radioactive source, for example a cobalt-60 source, with shielding that can be opened to provide a dose of radiation for radiotherapy, and closed between providing doses of radiation, for example during MRI imaging.

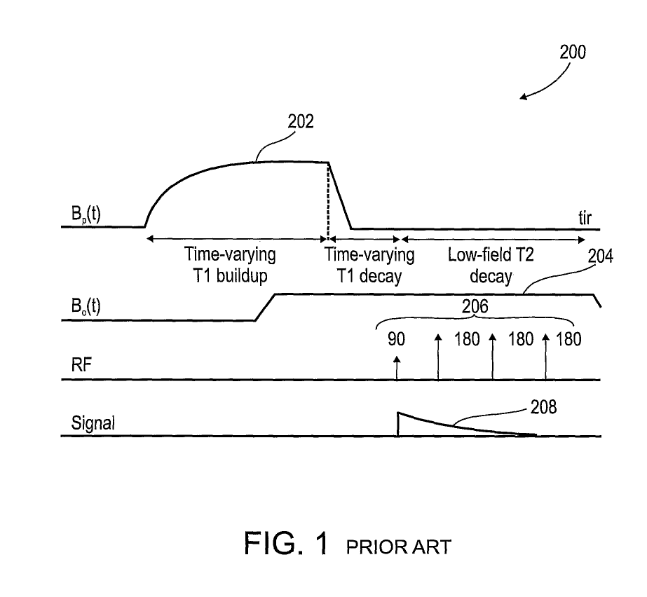

For purposes of better understanding some embodiments of the present invention, as illustrated in FIGS. 1-3 of the drawings, reference is first made to the construction and operation of a conventional (i.e., prior art) pulse sequence diagram 200 for a PMRI system as illustrated in FIG. 1. A high magnetic field 202 is ramped up, to polarize nuclei in a region being imaged. This polarization magnetic field need not be very uniform in space or time, since it is ramped down before readout of the image. A lower magnetic field 204, very uniform in the region being imaged, is used for readout of the image, during the application of 90 degree and 180 degree RF pulses 206, for example, which produce an NMR signal 208 from the polarized nuclei. Other MRI pulse sequences, well known to those skilled in the art of MRI can also be used to generate an MRI image.

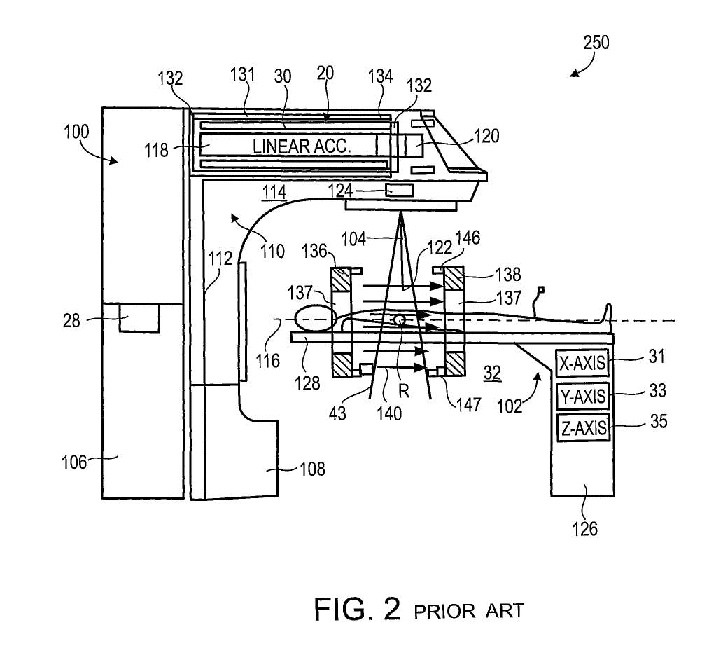

FIG. 2 shows a prior art combined MRI and radiotherapy system 250, using a linear accelerator 20 as a radiotherapy source, and an open MRI magnet comprising coils 136 and 138, generating a magnetic field 140 in a gap between them. Coils 136 and 138 are superconducting coils which remain on, producing a constant magnetic field, throughout the radiotherapy session. The linear accelerator produces a radiation beam 43 which irradiates a patient.

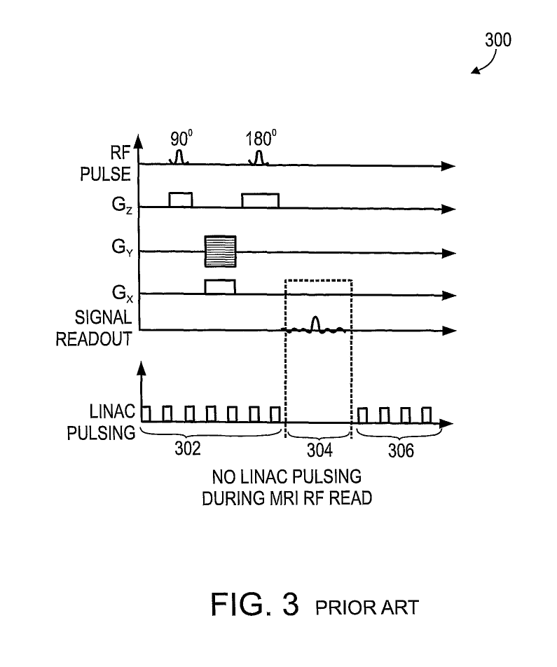

FIG. 3 shows a prior art timing diagram 300, for operation of such a combined MRI and radiotherapy system. During time interval 302, the linac is on, producing pulses of radiation, while RF and gradient pulses are produced by the MRI system. During time interval 304, the linac is turned off, and the NMR signal is read out by the MRI system. After readout, during interval 306, the linac is turned on again. In other prior art implementations, imaging and irradiation are not interleaved at all. Instead, they are performed sequentially, for example, a complete image is acquired, and a radiation dose then is applied.

Before explaining at least one embodiment of the invention in detail, it is to be understood that the invention is not necessarily limited in its application to the details of construction and the arrangement of the components and/or methods set forth in the following description. The invention is capable of other embodiments or of being practiced or carried out in various ways.

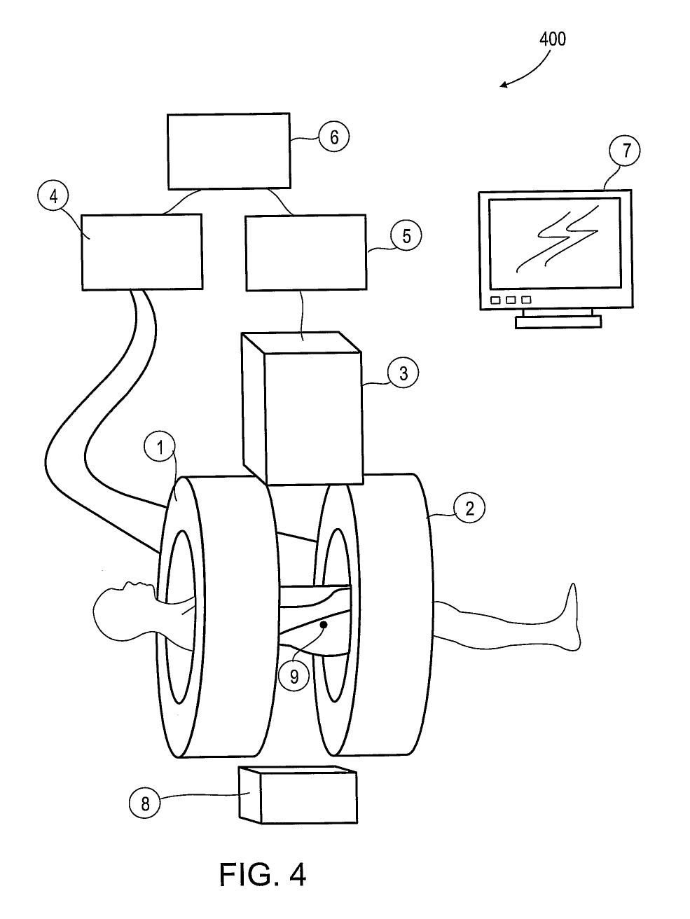

Referring now to the drawings, FIG. 4 illustrates a combined MRI and radiotherapy system 400, according to an exemplary embodiment of the invention. An MRI magnet, optionally incorporated together with gradient and RF coils, optionally has a superior segment 1 and an inferior segment 2, with an opening between through which radiation can pass unimpeded. A radiation source 3, optionally located just outside the MRI magnet adjacent to the opening, may be, for example, the head of a linac or a radioisotope source. The radiation source is optionally controlled by an irradiation control module 5 that governs whether radiation is emitted by the radiation source or not, for example by turning the linac on or off or by opening and closing the shielding for a radioisotope source. The operation of the MRI system is optionally controlled by an MRI control unit 4, which controls the typically incorporated gradient and the RF systems, as well as controlling the MRI magnet by ramping the magnet on or off. Also shown is an MRI-radiation synchronization unit 6 that optionally controls or synchronizes both the MRI system and the radiation source, allowing either one or the other to work, but not both simultaneously. Optionally, the functions of two or more of control units 4, 5 and 6 are performed by a single unit. Optionally, combined system 400 also comprises a display unit 7 for displaying MRI images and a real-time tracking system 8 that tracks the position, in real time, of a tumor receiving radiotherapy.

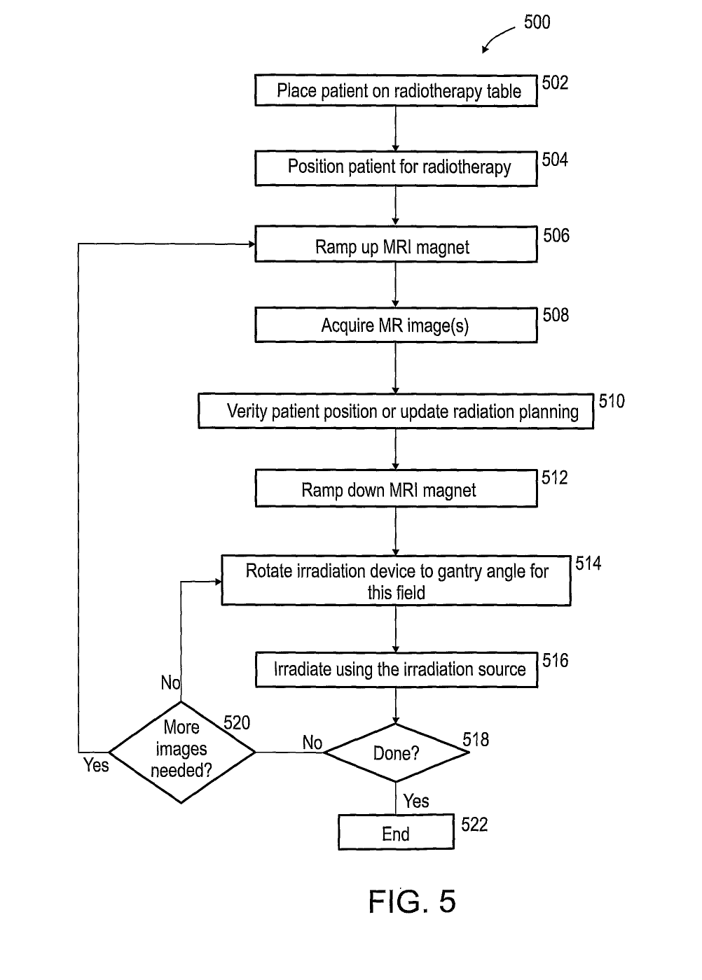

FIG. 5 shows a flow diagram 500 for a method of using a combined MRI and radiotherapy system, such as system 400. At 502, a patient is placed on a table of the combined system and, at 504, positioned in a proper position for radiotherapy and MRI. Optionally, for example if MRI imaging is desired for prior position verification or irradiation dose adjustment, the MRI magnet is ramped on at 506, and, once the magnetic field is stable, a set of one or more MRI images are acquired at 508. The resulting images are then optionally used, at 510, to verify the patient positioning for radiotherapy or to re-plan the radiation doses and/or gantry angles for the radiotherapy. At 512, the MRI magnet is then ramped down. Alternatively, the MRI magnet is ramped down before using the images. Optionally, when the magnet is ramped down, the magnetic field is less than 100 gauss in any volume in which the system is configured to receive part of the body of the patient, for example anywhere on the bed. Having such a low magnetic field everywhere in the patient's body may allow the magnetic field to be ignored in calculating the radiation dose. At 514, an irradiation gantry is moved to the angle, or position, of a first field for radiotherapy, as in a conventional radiotherapy system. Alternatively, the patient is moved to a different position relative to the irradiation gantry, for the first irradiation field, for example by moving the bed, but if this is done, the position of the patient remains well-defined relative to the gantry and the MRI system, since the bed, the gantry and the MRI system all remain registered to each other, with any relative motion between them well-defined. At 516, the patient is irradiated by the radiation source. At 518, a decision is made whether the radiotherapy has been completed. If not, at 520, a decision is made whether more MRI images are needed, for example to verify patient position before the next irradiation field. If more images are needed, control returns to 506, and the magnet is ramped up again. If no more images are needed at this point, then the radiation source is moved to the position for the next irradiation field, at 514. When all irradiation fields have been completed, the procedure ends at 520.

The system shown in FIG. 4 may be implemented in a variety of ways. Several exemplary variations are described below.
1) A first variation comprises a combination radiotherapy and MRI system wherein the MRI system includes a current-based (or non-permanent) MRI magnet. The combination system further comprises a timing component that coordinates the linac pulse such that the MRI magnet is off while the linac is irradiating and vice versa. The MRI magnet may be resistive or superconductive. In either case, the timing component ramps the MRI magnet down to a much lower field, optionally turning it off completely, as the linac irradiates the patient and then ramps the magnet back up to acquire an MRI image. Since the magnet is off or at a very low field during irradiation, the electron beam, for example, in the linac waveguide remains undisturbed and the dose distribution in the patient is unaffected, or is affected little enough not to matter, or is affected in a way that can be easily calculated and compensated for. MRI systems having permanent magnets as the main source of the MRI magnetic field are not suitable for this combination since the magnetic fields in such magnets are not rampable. For the same reasons, traditional high field superconducting magnets, with ramp times of several hours, are equally unsuitable for this variation, as the main source of the MRI magnetic field. However, one implementation of this first variation comprises high field superconducting magnets with short ramp time and another implementation comprises low field superconducting magnets with short ramp time. Some designs for such magnets are referenced below.
2) A second variation comprises a combination radiotherapy and MRI system wherein the MRI component comprises a high field MRI system. The high field MRI system component is a prepolarized (field cycled) MRI system, in which a high field polarizing magnetic field is cycled “on” and “off” during the MRI scan, “on” during the “spin preparation” phase, and “off” during the “readout” phase, while the homogeneous low-field magnet is used for sampling the MRI signal. As a result, this variation may have the resolution and signal-to-noise ratio advantages of high field MRI systems without many of the disadvantages. In this variation, the high-field magnet is resistive to allow its field to be ramped off before beginning linac irradiation. However, the low-field readout magnet may be either a resistive magnet or a superconducting magnet. Because low-field, rapidly-rampable, superconductive magnets are relatively easy to produce, as will be described below, they are a good choice for this variation.
3) A third variation comprises either of the first and second variations further in combination with a real-time tracker, as will be described in more detail below.
4) A fourth variation comprises a combination of a radioisotope-based radiotherapy component having a radiation source that is alternately shielded and exposed, and an MRI system component wherein the MRI magnet has any of the characteristics described above for the first and second variations.

In each of the variations described above, the MRI magnet optionally has an open design (for example a “double doughnut” such as the magnet shown in FIG. 2)) allowing the radiotherapy system component unimpeded access to the tumor. In addition, ancillary components such as MRI gradient and RF coils are optionally designed in such a way that they too allow for substantially unimpeded access by the radiotherapy radiation to the patient's target tissue, using, for example, the methods described in published PCT application WO 2006/097274, referenced above.

### Resistive Magnets

The potential drawbacks in resistive MRI magnets, described above at the end of the section “Field and Background of the Invention,” are much less problematic with our combined MRI and radiotherapy system. Since the magnet will be ramped up and down, the active duty cycle of the magnet is low, for example, 5 minutes out of every 20 minutes. As a result, the method for dispersing the heat generated by the magnet currents need not be highly efficient, or, for a given efficiency of heat removal, higher field can be achieved. In addition, since the MRI image is used only for treatment placement verification and for on-line updating of the treatment plan, the image quality and resolution may be somewhat lower than that used in diagnostic radiology. Thus, the image produced by a low field MRI system may be more than adequate for placement verification and for on-line updating of the treatment plan. Use of a low field system decreases the demands on the MRI system—less heat is generated in the magnet coils and it is easier to stabilize the magnet current. In addition, the demands on the magnet power supply are lower, decreasing the cost of the power supply and the cost of operating the magnet.

Integration of a resistive-magnet MRI system with a linac is potentially relatively straightforward and inexpensive. An example of a suitable magnet is that shown in the Proview MRI system produced by Picker International Inc. Details of this system, based on an open, iron-core, 0.23 Tesla electromagnet with a 44 cm patient gap, may be found in the presentation Interoperative MRI—New technology to Improve Neurosurgical Care by J Katisko, S Yrjänä,P Karinen, M Lappalainen,T Leppänen & J Koivukangas, presented at the 50^{th} annual meeting of the Scandinavian Neurosurgical Society, Oulu, Finland, Jun. 12-14, 1998. See www.oulu.fi/neurosurgeryinru/nru/poster, downloaded Nov. 22, 2007.

Another design for a suitable resistive magnet may be found in: An Open-Access, Very-Low-Field MRI System for Posture Dependent ³He Human Lung Imaging by L. L. Tasi, R. W. Mair, M. S. Rosen, S. Patz and R. L. Walsworth, to be published in J. Mag. Res (2007). An abstract is available on the web at: www.cfa.harvard.edu/Walsworth/Activities/Low%20field%20MRI/human_lowfield.ht ml, downloaded Nov. 22, 2007.

### Rapidly Rampable Superconducting Magnets

Other acceptable, rampable magnets include quickly rampable superconducting magnets. Generally, superconducting magnets used in clinical MRI are not ramped up and down, except in exceptional circumstances, such as those involving medical emergencies or servicing. The typical ramp-up time of a whole-body, high-field superconducting magnet is usually several hours to efficiently use electrical power, to minimize heat loads, to conserve liquid helium, and to stabilize the magnetic field. However, designs have been published for suitable superconducting magnets that can be ramped up more quickly.

One superconducting magnet design suitable for use in our combination device is exemplified in U.S. Pat. No. 5,838,995 (to Macovski and Conolly). Macovski et al describes a strong superconductive magnet that may be quickly pulsed. For example, Macovski et al discloses a device having a 5 Tesla field between a pair of magnet coils (a so-called “Helmholtz pair”) 20 cm. apart that may be ramped up or down in 200 msec without making unreasonable demands on the power supply or the energy storage capacitors. The Macovski superconductive magnet may be of a relatively small volume. Such a magnet may be especially suitable for tumor imaging, where the volume of interest is generally much more localized than for diagnostic imaging. U.S. Pat. No. 6,097,187 (to Srivastava et al and assigned to Picker International Inc.), entitled MRI Magnet with Fast Ramp Up Capability for Interventional Imaging, teach how to make a superconducting magnet that stabilizes almost immediately upon ramp up and may then be used for MRI. In these variations, the MRI system can be a high field system, with all the attendant advantages, viz. high signal-to-noise ratio (SNR), high resolution and fast imaging time.

### Shielded Magnets

Although the MRI magnetic field is not active during linac irradiation and therefore the path of the accelerated electrons is not affected by the MRI system, if the linac contains ferromagnetic materials, these materials may become magnetized by the magnetic field and this magnetization may remain present due to hysteresis, even when the MRI magnetic field is off. If this is a problem, the resistive magnet may be designed as a shielded magnet. Although the MRI magnets used in the 1970's and 80's were not shielded, the technologies used to shield superconducting magnets, active or passive shielding, may be used to ensure that the magnetic field in any part of the linac remains sufficiently low not to adversely affect the operation of the linac.

Using a shielded magnet also reduces the effect of any ferromagnetic material in the linac or other radiation source, or in any other nearby equipment, on the uniformity of the MRI magnetic field. Even small non-uniformity in the magnetic field during readout of the MRI signal can degrade the MRI image. Because the radiation source is generally moved in the course of radiotherapy treatment, it may be difficult to use shimming to compensate for any effect of ferromagnetic material in the radiation source on the uniformity of the MRI magnetic field, although active shimming may be possible.

### Prepolarized MRI System

Another variation of our combination device comprises a radiotherapy system with a high field MRI system. The resolution of MRI images is often limited by the signal-to-noise ratio, which is higher, for a given voxel size and acquisition time, for a higher magnetic field. The theoretical resolution of an MRI system may be very high, however, since the signal-to-noise ratio (SNR) decreases as the voxel size decreases, voxel sizes is often kept fairly large to ensure diagnostic-quality images. As a result, high field MRI systems usually attain higher SNR or higher resolution than low field MRI systems. Although low field strength may be sufficient for the purpose of treatment placement verification and for on-line updating of the treatment plan, there are substantial advantages to having a high resolution image. A high resolution image allows ease of tumor and organ boundary visualization. In addition, in a high field MRI system, resolution may be traded off for imaging time. A lower resolution image of nevertheless adequate quality may be acquired more quickly than in a low field system.

This variation comprising a PMRI system and a linac has the same advantages as does a high field system without having the high field constantly present. In most of the PMRI systems in use, the low field (or bias field) is operated constantly, since the low field is not rapidly rampable. However, in this variation, the bias field is also optionally rampable, and it is optionally ramped off at the end of an imaging session. Since the high field of the PMRI system is only pulsed on during the imaging sequence in any case, the magnetic field of the PMRI system does not interfere with the linac system at all in this case, except for possible magnetization of the linac, since imaging and radiation are not performed simultaneously.

Alternatively, the MRI system is a PMRI system with the low field magnet not quickly rampable, and left on continuously during a radiotherapy session. If the low field magnet is chosen to be low enough, its effect on the linac can be negligible. Similarly, the effect of a sufficiently low field on the radiation dose distribution is likewise negligible, as may be determined, for example, using the methods described in some of the papers by A. Raaijmakers et al, referenced above. Indeed, in PMRI the lower the readout magnetic field the better, up to a point. For example, the readout field is less than 1000 gauss, or less than 500 gauss, or less than 100 gauss.

System with Real-Time Tracker

Another variation of our combination device comprises an MRI-radiotherapy system further comprising a true real-time tracking system. Use of such a combination permits an even higher speed resolution of the position of the radiotherapy target. MRI systems may require at least 1-10 seconds to acquire a single diagnostic-quality image. Acquisition of the stack of images required for 3D reconstruction of the entire tumor may require at least a few minutes. On the other hand, real-time trackers track individual markers (for example 1 to 3 markers) in real-time, with negligible acquisition time.

Many real time tracker systems are suitable as a component of this variation. These systems utilize a variety of technologies such as RF tracking, radioactive marker tracking, camera-based tracking, etc. One suitable RF tracking system is described in U.S. Pat. No. 6,822,570, to Dimmer, Wright, and Mayo (assigned to Calypso Medical Technologies) which is based on excitation of an implanted leadless marker.

An example of a radioactive tracker is shown in Published PCT Application WO 2006/016368, to Kornblau and Ben Ari. Using such a tracker allows tracking of the tumor in real-time to verify that the patient is not moving or to track a tumor that moves due to respiration, peristalsis, etc. Such information may be used:
to verify that the tumor remains within the intended volume during the radiotherapy irradiation or
to keep the irradiation beam aimed properly if the patient shifts or otherwise moves during the radiotherapy irradiation,
to correct the aim of the irradiation beam if it moves (shifts or rotates) during the radiotherapy irradiation, or
to gate the irradiation beam, for example to the respiratory or cardiac cycle, to ensure that the tumor receives the required dose of radiation and healthy tissue is not irradiated any more than required.

Once a given irradiation field is complete, an MRI image can be acquired if necessary, to keep the real-time tracker accurately calibrated.

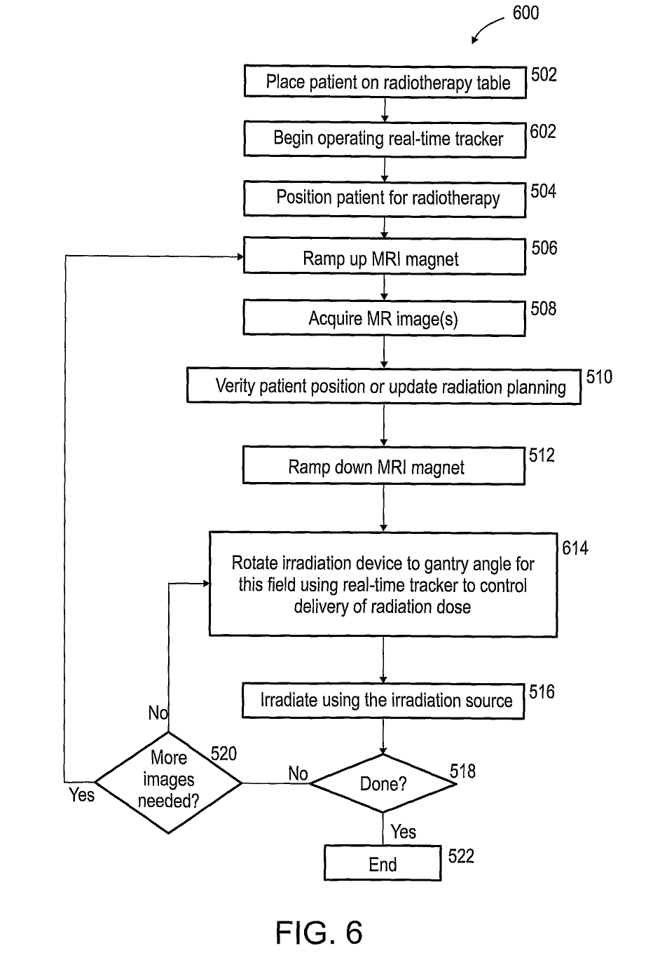

FIG. 6 shows a flow diagram 600 similar to flow diagram 500, but using a real-time tracker. The real-time tracker is optionally turned on at 602, after placing the patient on the table, or at any time before MRI images are acquired. At 614, if the radiation source is moved to a different gantry position, the gantry position is optionally set taking into account data from the real-time tracker, to measure any change in the position of the radiotherapy target, for example a tumor, since the last irradiation field. Optionally, data from the real-time tracker is also used at 520, in deciding whether more MRI images are needed. For example, more images may be acquired if the patient has moved so much that the real-time tracker may no longer provide an accurate estimate of the position of the target.

It is expected that during the life of a patent maturing from this application many relevant radiation sources for radiotherapy, and real-time trackers, will be developed and the scope of the terms radiation source, and real-time tracker, is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A combination MRI and radiotherapy system comprising:
a) an MRI system for imaging a patient receiving radiotherapy, comprising a magnetic field source suitable for generating a magnetic field of strength and uniformity useable for imaging, capable of being ramped up to said magnetic field in less than 10 minutes, and ramped down from said magnetic field in less than 10 minutes;
b) a radiation source configured for applying radiotherapy; and
c) a controller which ramps the magnetic field source down to less than 20% of said magnetic field strength when the radiation source is to be used for radiotherapy, and ramps the magnetic field source up to said magnetic field strength when the MRI system is to be used for imaging.

2. A system according to claim 1, wherein the magnetic field source comprises a non-superconducting coil.

3. A system according to any of the preceding claims, wherein the MRI system comprises a prepolarized MRI system, and the magnetic field source comprises a high field source for polarization and a low field source for readout.

4. A system according to claim 3, wherein the high field source comprises a superconducting coil capable of ramping up to a high magnetic field used for polarization, and ramping down from the high magnetic field, each in less than 10 minutes.

5. A system according to claim 3 or claim 4, wherein the low field source comprises a superconducting coil.

6. A system according to claim 5, wherein the superconducting coil of the low field source is capable of ramping up to a low magnetic field used for readout, and ramping down from the low magnetic field, each in less than 10 minutes.

7. A system according to any of the preceding claims, wherein the controller controls the MRI system not to acquire images when the radiation source is being used for radiotherapy.

8. A system according to any of the preceding claims, also comprising a real-time tracker which tracks changes in position of a radiotherapy target in the patient.

9. A system according to claim 8, wherein the tracker comprises an RF tracker.

10. A system according to claim 8 or claim 9, wherein the tracker includes a radioactive marker.

11. A system according to any of claims 8-10, wherein the tracker comprises an image-based tracking system.

12. A system according to any of claims 8-11, wherein the tracker comprises an implanted leadless marker.

13. A system according to any of the preceding claims, wherein the radiation source comprises a linac.

14. A system according to any of claims 1-13, wherein the radiation source comprises a radioactive source.

15. A system according to any of the preceding claims, wherein the magnetic field source of the MRI system comprises an open magnet.

16. A system according to any of the preceding claims, wherein the magnetic field source is sufficiently well shielded magnetically such that the magnetic field used for imaging is less than 100 gauss throughout any volume where the radiation source is located during imaging.

17. A system according to any of the preceding claims, wherein the controller and magnetic field source are configured such that when the controller ramps the magnetic field source down, the magnetic field is less than 100 gauss in any volume in which the system is configured to receive part of the body of the patient during radiotherapy.

18. A system according to any of the preceding claims, wherein the magnetic field used for imaging reaches at least 1 tesla, throughout an imaging region, when the magnetic field source is ramped up.

19. A method of radiotherapy of a target volume in a patient, comprising:
a) ramping up a magnet of an MRI system in less than 10 minutes;
b) acquiring one or more MRI images of the target volume, using the ramped up magnetic field;
c) ramping down the magnet to a field lower by at least a factor of 5, in less than 10 minutes, after using the field for the MRI imaging; and
d) applying radiotherapy radiation from a radiation source to the target volume with the magnet ramped down, taking into account the position of the target volume as indicated in the MRI images, while keeping the radiation source registered to the MRI system between acquiring the images and applying the radiation.
